(19) United States
(12) Patent Application Publication
(10) Pub. No.: US 2008/0044813 A1
(43) Pub. Date: Feb. 21, 2008

(54) Amp Ligation Assay (Ala)

(76) Inventors: Kristian Jansson, Jyvaskyla (FI); Vuokko Jansson, Jyvaskyla (FI)

Correspondence Address: Kristian Jansson, Aumakatu 3, Jyvaskyla, FIN-40320, omitted

(21) Appl. No.: 10/550,541

(22) PCT Filed: Mar. 20, 2003
(86) PCT No.: PCT/FI03/00216

§ 371 Date: Apr. 10, 2007

(51) Int. Cl. C12Q 1/68; C12Q 1/66

(52) U.S. Cl. 435/6; 435/8

(57) ABSTRACT

The present invention relates to a method, reagent and kit for detecting ligase-catalyzed joining of nucleic acid ends, and any analyte related thereto, such as a ligase, or a substrate or cofactor thereof. More specifically, the invention relates to a method and kit for detecting the presence or amount of a target nucleic acid sequence in a sample. In the inventive method, ligase-catalyzed joining of nucleic acid ends is detected by detecting the AMP released thereby, preferably by using enzymatic means that comprises luciferase and luciferin.

The present invention relates to a method, reagent and kit for detecting ligase-catalyzed joining of nucleic acid ends, and any analyte related thereto, such as a ligase, or a substrate or cofactor thereof. More specifically, the invention relates to a method and kit for detecting the presence or amount of a target nucleic acid sequence in a sample.

Ligases are enzymes that catalyze the covalent joining of adjacent nucleic acid ends by forming phosphodiesterase bonds between 5′-phosphate and 3′-hydroxyl groups. DNA ligases such as DNA ligase (ATP) and DNA ligase (NAD) catalyze the formation of phosphodiester bonds at the site of single-stranded breaks (nicks) in double-stranded DNA. They also catalyze the formation of phosphodiester bonds in double-stranded DNA containing complementary cohesive ends that base pair to bring together 3′-hydroxyl and 5′-phosphate groups. Blunt-ended DNA duplexes containing 5′-phosphate and 3′-hydroxyl groups can serve as substrates for DNA ligases, at high concentrations. RNA ligase (ATP) catalyzes joining of single-stranded RNA (or DNA) molecules containing 5′-phosphate and 3′-hydroxyl groups.

The reaction catalyzed by DNA ligases using either ATP or NAD as a cofactor involves three reversible steps. First, the enzyme is activated through the formation of a covalent ligase-adenylate intermediate with concomitant release of pyrophosphate (from ATP) or nicotinamide mononucleotide (from NAD). In the second step, the adenylate moiety is transferred to the 5′-phosphate group at the single-strand break site. Finally, a phosphodiester bond is formed by a nucleophilic attack of the adjacent 3′-hydroxyl group on the adenylated 5′-phosphate group with concomitant release of AMP.

Mismatches close to the single-strand break site inhibit the ligation reaction. This is the basis for ligase-mediated detection of target DNA (or RNA) sequences to distinguish allelic sequence variants such as mutations and single-nucleotide polymorphisms, or to detect exogenous nucleic acid sequences such as those from pathogenic bacteria and viruses.

In oligonucleotide ligation assays (Landegren et al., Science 241: 1077-80 (1988); U.S. Pat. No. 4,883,750; U.S. Pat. No. 4,988,617; WO 95/22623), two nucleic acid probe sequences are hybridized to two adjacent regions of a target nucleic acid sequence such that the 5′-phosphate group of one probe sequence abuts the 3′-hydroxyl group of the other. The probe sequences may be designed to hybridize to the target sequence to leave a gap of one or more nucleotides between the sequences, which gap is then filled by an extension reaction. The probe sequences may be two separate oligonucleotides at least one of which is labelled or the two free ends of a single labelled oligonucleotide. The label may be radioactive, fluorescent, antigenic, or the like. If there are no mismatches close to the nick, the probe sequences can be joined by a ligase. The hybridizing and ligating steps may be repeated one or more times. The ligated product is first separated from the labelled oligonucleotides and then detected by the label, wherein the presence or absence of the ligated product is indicative of the presence or absence of the target sequence.

Therefore, one object of the present invention is to provide a ligase-mediated method for detecting a nucleic acid target sequence, in which method neither labelling of the nucleic acid probe sequences nor the separation of the ligated product is necessary. Another object of the present invention is to provide a method for detecting ligase-catalyzed joining of nucleic acid ends by non-radioactive, enzymatic means.

Thus, the invention in one embodiment provides a method for detecting a target nucleic acid sequence in a sample, which method comprises: (a) providing two nucleic acid probe sequences which are at least partially complementary to and capable of hybridizing to two adjacent regions of said target sequence; (b) hybridizing said probe sequences to said target sequence under hybridizing conditions; (c) joining said probe sequences with a ligase; d) optionally repeating the steps (b) and (c) one or more times; and (e) detecting the AMP released; wherein the presence or amount of the AMP released is indicative of the presence or amount of said target sequence. The method also contemplates interrogating the presence or absence of a specific base in a nucleic acid target sequence in a sample to be assayed.

In another embodiment, the invention provides a kit for use in the above method, which kit comprises in a packaged combination: (a) a ligase, and (b) AMP detecting means.

In yet another embodiment, the invention provides a method for detecting ligase-catalyzed joining of nucleic acid ends, which method comprises detecting by enzymatic means the AMP released.

In yet another embodiment, the invention provides a reagent for detecting ligase-catalyzed joining of nucleic acid ends, which reagent comprises enzymatic means for detecting the AMP released.

In yet another embodiment, the invention provides a kit for detecting ligase-catalyzed joining of nucleic acid ends, which kit comprises, in a packaged combination, enzymatic means for detecting the AMP released.

By detecting is meant detecting the presence or amount.

Appropriate conditions for the method of the present invention include appropriate component concentrations, solution temperature, ionic strength, and incubation time. Such conditions also include the presence of any appropriate additional substances, such as enzyme activators, cofactors, stabilizers, and buffering agents. Appropriate incubation conditions for a given enzyme, or coupled enzyme system, are generally known in the art or are readily determined using standard methods known in the art.

In preferred embodiments the ligase is a DNA ligase, and in particular, DNA ligase (NAD) such as Escherichia coli DNA ligase available from New England Biolabs (Beverly, Mass.) and from Amersham Biosciences (Piscataway, N.J.). Thermostable Thermus scotoductus (Tsc) DNA ligase (NAD) is available from Roche (Mannheim, Germany).

By amplification is meant the increase in the number of copies of a particular nucleic acid sequence. A nucleic acid target sequence of any origin (human, animal, plant, bacterial, viral, etc.) may be amplified to produce a detectable number of copies thereof. The polymerase chain reaction (PCR) is one of the routine methods of amplification (see, for references, Saiki et al., Science 239: 487-91 (1988); Sambrook and Russel, eds., Molecular Cloning: A Laboratory Manual, third edition, Cold Spring Harbor Laboratory Press, Cold Spring Harbor, N.Y., (2001)).

For imrnmobilization, the target sequence is preferably amplified using PCR with a biotinylated oligonucleotide primer to produce a biotinylated amplification product in which the biotin moiety is attached to the strand comprising the target sequence. The biotinylated amplification product is then bound to a streptavidin-coated solid support and subjected to strand separation. The immobilized single-stranded target sequence is detected by the method of invention.

Biotinylated and other modified as well as unmodified oligonucleotides are available from a number of companies, for example, from TAG Copenhagen, Copenhagen, Denmark.

As the solid support, superparamagnetic beads (ricrospheres) with covalently bound streptavidin, such as Dynabeads M-280 Streptavidin available from Dynal, Oslo, Norway, are preferred. Such beads offer easy manipulations and the possibility of working at kinetic rates close to those occurring in free solutions. See, for references, Hultman et al., Nucleic Acids Res. 17: 4937-46 (1989); McPherson, ed., PCR 2—A Practical Approach, IRL Press at Oxford University Press, Oxford, UK (1995).

In preferred embodiments, the AMP released is detected by a luciferase-luciferin reaction after converting it to ATP. In the presence of ATP and 02, luciferase catalyzes the oxidation of luciferin, producing light. Additional products of the reaction are AMP, pyrophosphate and oxyluciferin. The light can be detected by a luminometer or similar light-sensitive instrument, or more specifically, by a photomultiplier, a photodiode, a charged coupled device (CCD), or the like. Preferred luciferase is recombinant firefly luciferase available for example from Promega (Madison, Wis.) and, as a kit component, from Molecular Probes (Eugene, Oreg.).

In particularly preferred embodiments, the AMP released is converted to ATP by means of adenylate kinase, nucleoside-diphosphate kinase, and dCTP (2′-deoxycytidine 5′-triphosphate) or another phosphate donor, as described in our copending application PCT/FI03/00131.

In some embodiments, AMP is detected enzymatically without converting it to ATP. For example, AMP can be detected by using it to stimulate the activity of added glycogen phosphorylase which converts added glycogen and inorganic phosphate into glucose-1-phosphate. In the presence of phosphoglucomutase and glucose-6-phosphate dehydrogenase, glucose-1-phosphate is converted first to glucose-6-phosphate and then to 6-phosphogluconolactone in a reaction in which added NADP is converted to NADPH which is detected by UV absorbance (340 nm) or fluorescence emission (460 nm). This AMP detection system is exemplified in U.S. Pat. No. 5,316,907, incorporated herein by reference.

In an alternative embodiment, the AMP released is fragmented to ions and detected by its mass spectra using a technique such as desorption/ionization on silicon (DIOS) by Wei et al., Nature, 399: 243-246 (1999) that can accurately perform assays on picogram amounts using commercially available mass spectrographs adapted with a specialized porous silicon sample well. The older, well known, MALDI mass spectrographic assay techniques can also be utilized. For discussion and examples of mass spectrometric nucleotide analyses see U.S. Pat. No. 6,235,480, incorporated herein by reference.

The following examples are intended to illustrate the present invention and in no way limit any aspect of the invention.

### EXAMPLE 1

Reaction Buffer:

25 mM aqueous Tricine buffer, pH 7.8, 5 mM MgSO₄, 0.1 mM EDTA, and 0.1 mM sodium azide (Molecular Probes, Component E of A-22066, Lot 64B1-1).

Reagent A:

200 μM dCTP (sodium salt, Amersham Biosciences, 272062, Lot 8620),

20 units/ml myokinase (Sigma, M5520, Lot 012K7485),

20 units/ml nucleoside-diphosphate kinase (Sigma, N0379, Lot 119H7455),

50 units/ml E. coli DNA ligase (New England Biolabs, M0205S, Lot 44A), and

20 μM NAD (β-nicotinamide adenine dinucleotide, Sigma, N1511, Lot 110K7073) in Reaction Buffer.

Reagent B:

5 mM D-luciferin (sodium salt, Molecular Probes, Component A of A-22066, Lot 64B1-1),

50 μg/ml luciferase, firefly recombinant (Molecular Probes, Component B of A-22066, Lot 64B1-1), and

10 M DTT (dithiothreitol, Molecular Probes, Component C of A-22066, Lot 64B 1-1) in Reaction Buffer.

Reagent C:

45 volumes of Reagent A, and

10 volumes of Reagent B.

### EXAMPLE 2

A sample containing 0.1-10 pmol AMP (sodium salt, Signa, A1752, Lot 042K7000) or 10 pmol ATP (disodium salt, Molecular Probes, Component D of A-22066, Lot 64B1-1) in 45 μl of Reaction Buffer was mixed in a polystyrene test tube (Sarstedt, 55476) with 55 μl of Reagent C except that E. coli DNA ligase and NAD were omitted, and after 10, 20 and 30 min at room temperature, the tube was read in a Berthold 9509 luminometer for 10 s. RLU=relative light units. No-analyte control (blank) was included. The average readings for duplicate reactions are shown in Table 1.

TABLE 1

| | Average RLU | | |
|---|---|---|---|
| AMP (pmol) | 10 min | 20 min | 30 min |
| — | 512 | 510 | 500 |
| 0.1 | 900 | 890 | 878 |
| 0.5 | 2460 | 2274 | 2004 |
| 1 | 4616 | 4396 | 3772 |
| 5 | 17802 | 17814 | 16730 |
| 10 | 34874 | 33200 | 32031 |
| ATP (10 pmol) | 35456 | 33659 | 31268 |

Table 1 shows that AMP in a sample can be detected in a single step with Reagent C (without E. coli DNA ligase and NAD). The light signal is directly proportional to the quantity of AMP and substantially constant for at least 30 minutes.

### EXAMPLE 3

A partial sequence (bases 12-125) of the human HBB gene normal (A) allele and that of the human HBB gene sickle-mutant (S) allele (base 70, A→T) were synthesized and used as model targets. The variant base is underlined.

Allele sequence A (synthetic single-stranded DNA oligonucleotide):

5′TGACACAACTGTGTTCACTAGCAACCTCAAACAGACACCATGGTGCAC
CTGACTCCTGAGGAGAAGTCTGCCGTTACTGCCCTGTGGGGCAAGGTGAA
CGTGGATGAAGTTGGT3′

Allele sequence S (synthetic single-stranded DNA oligonucleotide):

5′TGACACAACTGTGTTCACTAGCAACCTCAAACAGACACCATGGTGCAC
CTGACTCCTGTGGAGAGTCTGCCGTTACTGCCCTGTGGGGCAAGGTGAAC
GTGGATGAAGTTGGT3′

Probe sequence P-A is fully complementary with allele sequence A, but is mismatched with allele sequence S at the variant base position. Probe sequence P-S is fully complementary with allele sequence S, but is mismatched with allele sequence A at the variant base position. Probe sequence P (5′-phosphorylated) is fully complementary with both allele sequences. It hybridizes immediately adjacent to P-A or P-S and is suitable for ligation with either of them if there is no mismatch at the variant base position (at the ligation junction).

Probe sequence P-A (synthetic single-stranded DNA oligonucleotide):

5′CAGTAACGGCAGACTTCTCCT3′

Probe sequence P-S (synthetic single-stranded DNA oligonucleotide):

5′CAGTAACGGCAGACTTCTCCA3′

Probe sequence P (synthetic single-stranded DNA oligonucleotide, 5′-phosphorylated):

5′CAGGAGTCAGGTGCACCATG3′

All oligonucleotides were synthesized and purified by TAG Copenhagen.

### EXAMPLE 4

A sample containing 0.5-5 pmol of allele sequence S was admixed with 5 pmol allele sequence A, 50 pmol of probe P and 50 pmol of probe P-S in a thin-walled 200-μl tube (Roche, 1667041) in 50 μl of Reaction Buffer. The admixture was heated to 65° C. for 5 min and then allowed to cool at room temperature for 30 min. Then, 45 μl of the admixture was mixed in a polystyrene test tube (Sarstedt, 55476) with 55 μl of Reagent C, and after 10, 20 and 30 min at room temperature, the tube was read in a Berthold 9509 luminometer for 10 s. RLU=relative light units. Appropriate controls were included. The average readings for duplicate reactions are shown in Table 2.

TABLE 2

| | |
|---|---|
| Allele sequence | |
| (pmol) | Average RLU |

| S | A | 10 min | 20 min | 30 min |
|---|---|---|---|---|
| — | — | 1210 | 1117 | 1065 |
| — | 4.5 | 1426 | 1379 | 1408 |
| 0.45 | 4.5 | 2926 | 2946 | 2794 |
| 0.9 | 4.5 | 4200 | 4078 | 4024 |
| 2.25 | 4.5 | 7928 | 7938 | 7665 |
| 4.5 | 4.5 | 13956 | 13912 | 13765 |

Table 2 shows that a target sequence in a sample can be detected in a single step with Reagent C using the method of the invention. The light signal is directly proportional to the quantity of that sequence and substantially constant for at least 30 minutes. With the method of the invention, fmol quantities of a target sequence can be detected even in the presence of another sequence which differs from the target sequence only at a single base position.

### EXAMPLE 5

A sample containing 2.5 pmol of both allele sequences A and S or 5 pmol of either allele sequence A or S was admixed with 50 pmol of probe P and 50 pmol of either probe P-A or probe P-S in a thin-walled 200-μl tube (Roche, 1667041) in 50 μl of Reaction Buffer. The admixture was heated to 65° C. for 5 min and then allowed to cool at room temperature for 30 min. Then, 45 μl of the admixture was mixed in a polystyrene test tube (Sarstedt, 55476) with 55 μl of Reagent C, and after 10, 20 and 30 min at room temperature, the tube was read in a Berthold 9509 luminometer for 10 s. RLU=relative light units. Appropriate controls were included. The average readings for duplicate reactions are shown in Table 3.

TABLE 3

| Allele | | | | | | |
|---|---|---|---|---|---|---|
| sequence | | | | | | |
| (pmol) | | Probe | | Average RLU | | |
| A | S | P-A | P-S | 10 min | 20 min | 30 min |
| — | — | + | − | 1331 | 1290 | 1248 |
| — | — | − | + | 1292 | 1239 | 1186 |
| 4.5 | — | + | − | 16306 | 15902 | 15821 |
| 4.5 | — | − | + | 1210 | 1234 | 1198 |
| — | 4.5 | + | − | 1381 | 1400 | 1420 |
| — | 4.5 | − | + | 13333 | 13394 | 13054 |
| 2.25 | 2.25 | + | − | 7898 | 8423 | 8644 |
| 2.25 | 2.25 | − | + | 7228 | 6558 | 6237 |

Table 3 shows that the method of the invention can discriminate between homozygotes for these alleles, and can be used to detect heterozygote samples in which both alleles are present together, as would be the case with a carrier for a wide variety of genetic diseases. The results also show that the method of the invention can be used for interrogating the presence or absence of a specific base in a nucleic acid target sequence in a sample to be assayed. Note, that the signal is substantially constant for at least 30 min, which permits automated detection.

1. A method for detecting a target nucleic acid sequence in a sample, characterized in that it comprises: (a) providing two nucleic acid probe sequences which are at least partially complementary to and capable of hybridizing to two adjacent regions of said target sequence; (b) hybridizing said probe sequences to said target sequence under hybridizing conditions; (c) joining said probe sequences with a ligase; d) optionally repeating the steps (b) and (c) one or more times; and (e) detecting the AMP released; wherein the presence or amount of the AMP released is indicative of the presence or amount of said target sequence.

2. A method according to claim 1, wherein said probe sequences hybridize to said target sequence to leave a gap of one or more nucleotides between adjacent probe sequences, and wherein said step (b) further comprises filling said gap by an extension reaction prior to joining said probe sequences.

3-4. (canceled)

5. A method according to claim 1 or 2, wherein the AMP released is detected by enzymatic means.

6. A method according to claim 1 or 2, wherein the AMP released is detected by enzymatic means comprising luciferase and luciferin.

7. A method according to claim 1 or 2, wherein the AMP released is detected by enzymatic means comprising adenylate kinase, nucleoside-diphosphate kinase, dCTP or another phosphate donor, luciferase, and luciferin.

8. A method according to claim 1 or 2, wherein said target sequence is a DNA or RNA sequence.

9. A method according to claim 1 or 2, wherein said two probe sequences are within two separate oligonucleotides.

10. A method according to claim 1 or 2, wherein said two probe sequences are the two free ends of a single oligonucleotide.

11. A method according to claim 1 or 2, wherein said target sequence is in a single-stranded form.

12. A method according to claim 1 or 2, wherein said target sequence is an amplification product.

13. A method according to claim 1 or 2, wherein at least one of said probe sequences is immobilized to a solid phase.

14. A method according to claim 1 or 2, wherein said target sequence is immobilized to a solid phase.

15. A kit for use in a method according to claim 1 or 2, characterized in that it comprises in a packaged combination: (a) a ligase, and (b) AMP detecting means.

16-17. (canceled)

18. A kit according to claim 15, wherein said AMP detecting means is enzymatic means.

19. A kit according to claim 15, wherein said AMP detecting means is enzymatic means comprising luciferase and luciferin.

20. A kit according to claim 15, wherein said AMP detecting means is enzymatic means comprising adenylate kinase, nucleoside-diphosphate kinase, dCTP or another phosphate donor, luciferase, and luciferin.

21. A method for detecting ligase-catalyzed joining of nucleic acid ends, characterized in that it comprises detecting by enzymatic means the AMP released.

22. A method according to claim 21, wherein said ligase is a DNA ligase.

23. A method according to claim 21, wherein said ligase is DNA ligase (NAD).

24-26. (canceled)

27. A method according to claim 21, wherein said enzymatic means comprises luciferase and luciferin.

28. A method according to claim 21, wherein said enzymatic means comprises adenylate kinase, nucleoside-diphosphate kinase, dCTP or another phosphate donor, luciferase, and luciferin.

29-44. (canceled)
